(19) United States
(12) Patent Application Publication
(10) Pub. No.: US 2002/0076059 A1
(43) Pub. Date: Jun. 20, 2002

(54) Apparatus and method for reducing noise

(76) Inventor: George Malcolm Swift Joynes, Chilworth (GB)

Correspondence Address: EVENSON, McKEOWN, EDWARDS & LENAHAN, P.L.L.C., Suite 700, 1200 G Sreet, N.W., Washington, DC, 20005, US

(21) Appl. No.: 09/821,118

(22) Filed: Mar. 30, 2001

(30) Foreign Application Priority Data
Mar. 30, 2000 (GB) GB 007590.3

(51) Int. Cl. A61F 11/06; G10K 11/16; H03B 29/00

(52) U.S. Cl. 381/71.6; 381/71.4; 381/86

(57) ABSTRACT

In accordance with the present invention, an apparatus and method for reducing noise in a vehicle is provided. The apparatus uses an interferometer disposed in a headset to measure the sound proximate the driver's ears. An equal and opposite sound is generated and transmitted to the ear thereby reducing the level of noise audible to the driver. A microphone may be provided to make a separate measurement of the background noise to facilitate the noise reduction. A tracking device may be used to find and track the location of the driver's ears.

The present invention relates to the field of noise reduction. More specifically, the present invention relates to apparatus and a method for reducing noise in a vehicle.

The term noise can be used to refer to unwanted signals in general. These unwanted signals can take many forms, such as electromagnetic signals or sound waves. From hereinafter in this document the term noise is used to refer to unwanted sound waves.

Background noise is present almost everywhere. As town and cities become more crowded, background noise levels are reaching unprecedented levels causing people to take extreme measures to reduce the noise. Sound reducing fencing is being used along motorways to reduce noise caused by road traffic. Double-glazed windows are used in most modem houses to reduce street noise.

Similarly, background noise is a problem for drivers of cars and lorries. This noise comes from several sources, including the road, the vehicle's engine, other vehicles, and the wind. Means for reducing this background noise has been investigated for a considerable period of time, with limited success.

The two main approaches to noise reduction in general can be classified as passive and active methods.

Passive noise reduction methods use design and material technology to reduce the amount of noise being emitted from the source. Passive methods also includes absorbing or reflecting what remains of the noise to minimise the amount that is audible to, for example, the vehicle's driver and passengers. A further passive method is to cover or seal a person's ears with, for example, ear plugs. The passive approach to noise reduction has had some success, albeit at considerable expense. Furthermore, passive noise reduction methods put undesirable limitations on the vehicle's design and may be inconvenient to the user.

The second approach to noise reduction is active noise control. This involves the use of acoustic transducers and electronic systems to create spaces, usually of a limited size, within which the noise is sampled and an equal and oppositely polarised cancellation sound is introduced. Both feedback and feedforward techniques can be used to achieve active noise cancellation. The key requirements to achieving good active noise cancellation are either to be able to measure the noise in the vicinity of the cancellation space or derive an accurate estimate of the noise by an independent means, to generate an accurate replica of the noise, and to deliver this replica to the cancellation space without exacerbating noise problems in other areas.

It is known that active noise control can be achieved over a volume that is small in relation to the wavelength of the interfering sound. Thus for a narrowband noise signal, the carcelling signal is opposite in phase with the interfering sound. As the distance between the point of maximum cancellation and the sample point increases, the phase difference moves from 180 degrees and a very rapid fall off in cancellation effectiveness occurs either side of the 180 degree point. The same principle applies to a wideband noise signal, although the worst performance will be achieved at the highest end of the frequency band.

The amount of cancellation that is needed will vary between applications. For example, human hearing considers a reduction of 10 dB (decibel) in sound level to correspond to a subjective halving of the sound level. Thus, a 20 dB reduction in sound will provide a significant and noticeable reduction in noise. However, to achieve this over a bandwidth of 3 kHz requires the sample point to be no more than 1.7 mm from the point of maximum cancellation. This present a technical problem.

Currently, this technical problem of having to place the sensor close to the cancellation point has been addressed in two ways. The sensing device, such as a microphone, can be placed very close to the volume where maximum cancellation is required. Alternatively, predictive techniques are used which allow several measurements to be made away from this volume.

The first of these known approaches suffers from the need to mount a microphone obtrusively with respect to the cancellation volume. For instance, if the noise reduction is to be achieved for normal audio frequencies in a passenger's ears, the microphones have to be placed very close to the ear canal of each ear. Ideally, the cancellation should take place at the eardrum itself, but it is thought that because the canal is like a waveguide, with little dispersion, it is sufficient to do the cancellation at the entrance to the ear canal. However, this approach is often unacceptable due to the obtrusive nature of the microphones.

The second of these known ways is to use predictive techniques which enable the sensing of the sound signal to be made at a point or points remote from the cancellation volume. The problem with this approach is that the ability to predict the required cancellation signal is severely influenced by any changes in the geometry or characteristics of the environment, and thus will only work in rather simple and well controlled environments.

Thus, there is a need to find a way of achieving a consistently high degree of cancellation over as much of the audio band as possible, using a technique that does not require mechanical access to the entrance to the ear canal, or use unreliable predictive techniques.

It is an object of the present invention to provide a reliable apparatus and method for reducing noise in a vehicle, which is both cost effective and is not obtrusive to the driver or passengers of the vehicles.

It is a further object of the present invention to provide an apparatus and method for reducing noise in a vehicle which relies on remotely measuring the sound pressure variations in the vicinity of the ear canal in order to obtain the information needed to generate a noise cancellation signal.

According to the present invention there is provided apparatus for reducing noise in an area, comprising a transducer disposed at a first location and arranged to transduce sound substantially in said area into a signal which is measurably by a measuring device, said measuring device being disposed at said first location or at a second location and coupled to a sound cancellation device, said sound cancellation device being configured to generate a cancellation signal of approximately an equal intensity and opposite polarity to the said measurably signal and to transmit said cancellation signal to said area thereby substantially reducing the amount of noise audible in said area.

The first location may be in or proximate to said area.

The second location may be remote from said area.

The predetermined location may be proximate to a human ear.

The transducer may be human skin. The skin may be part of a human pinna. The skin may be the concha. The skin may be the cavum.

The transducer may be painted with pressure sensitive paint.

The transducer may be a sensor. The sensor may be arranged to generate a voltage in response to sound. Alternatively, the sensor may be arranged to generate a magnetic field in response to sound.

The measuring device may be an optical device, such as an interferometer. The interferometer may use a laser as a light source.

The measuring device may be arranged to measure a voltage. Alternatively, the measuring device may be arranged to measure a magnetic field.

The apparatus may further comprise a tracking device arranged to search for said transducer, to acquire a location of said transducer, and to track said location of said transducer, said tracking device being further arranged to communicate said location of said transducer to said measuring device.

The tracking device may be disposed in a headrest. The tracking device may be a video tracking device.

The apparatus may further comprise a further measuring device disposed remote to said area and arranged to measure background noise remote to said area, said background noise being communicated to said sound cancellation device to facilitate reducing the amount of noise audible in said area.

The further measuring device may be a microphone.

The apparatus may further comprise a filter disposed between said measuring device and said cancellation device and arranged to pass a range of frequencies, thereby enabling said apparatus to cancel noise based on a frequency of said noise.

The area may be in a vehicle.

According to the present invention there is provided a method for reducing noise in an area, the method comprising the steps of transducing sound in said area into a signal, measuring said signal from a location remote to said area, generating a signal of approximately an equal intensity and opposite polarity to said measured signal, and transmitting said generated signal to said area, thereby substantially reducing the amount of noise audible in said area.

The method may comprise the further step of measuring background sound remote to said area, and using said measurement of background sound to facilitate the reducing the amount of noise audible in said area.

While the principal advantages and features of the invention have been described above, a greater understanding and appreciation of the invention may be obtained by referring to the drawings and detailed description of a preferred embodiment, presented by way of example only, in which;

FIG. 1 is a diagram of the preferred embodiment of the present invention,

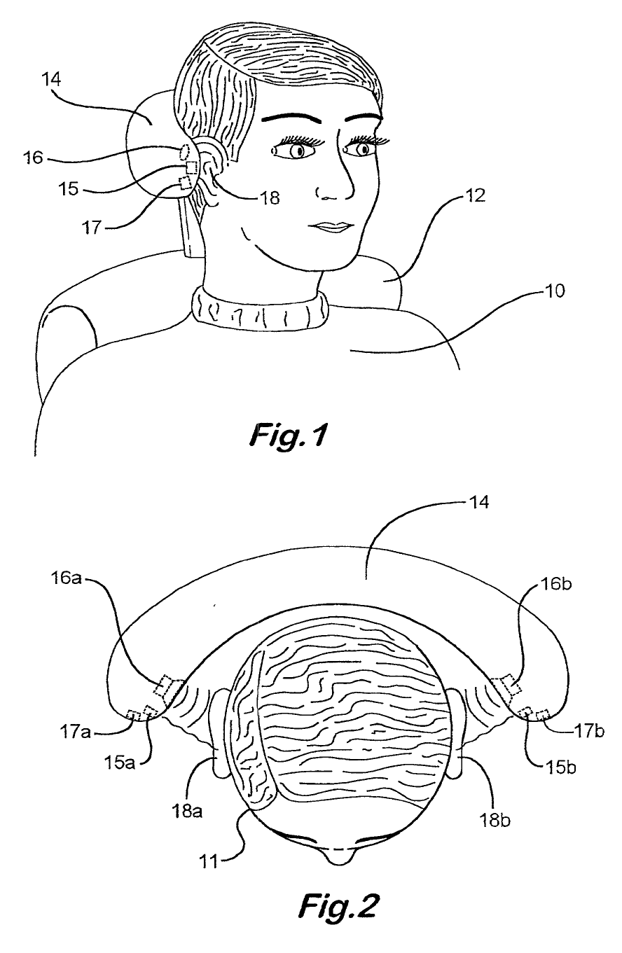

FIG. 2 is a top view of the embodiment shown in FIG. 1,

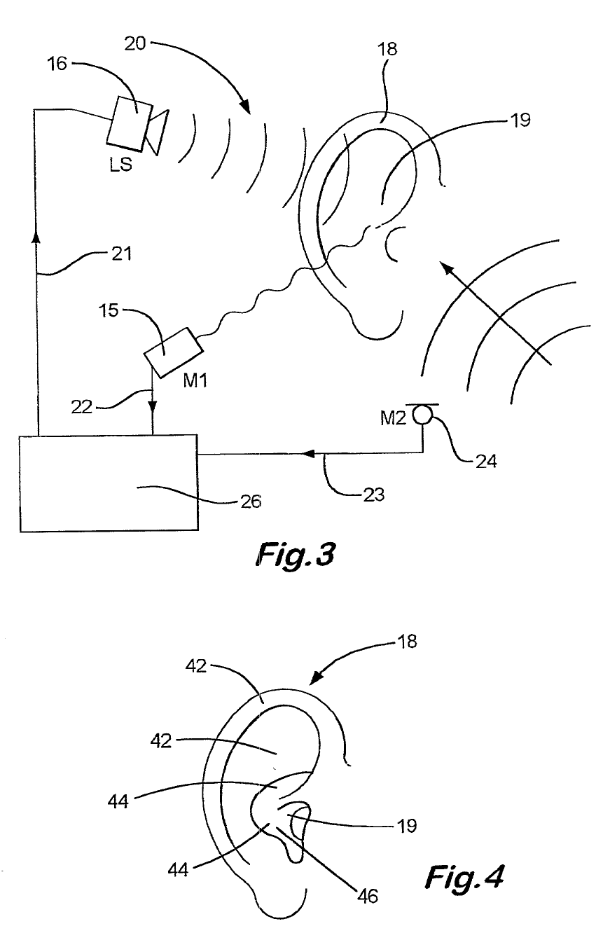

FIG. 3 shows the basic architecture of the preferred embodiment,

FIG. 4 shows some of the parts of a human ear,

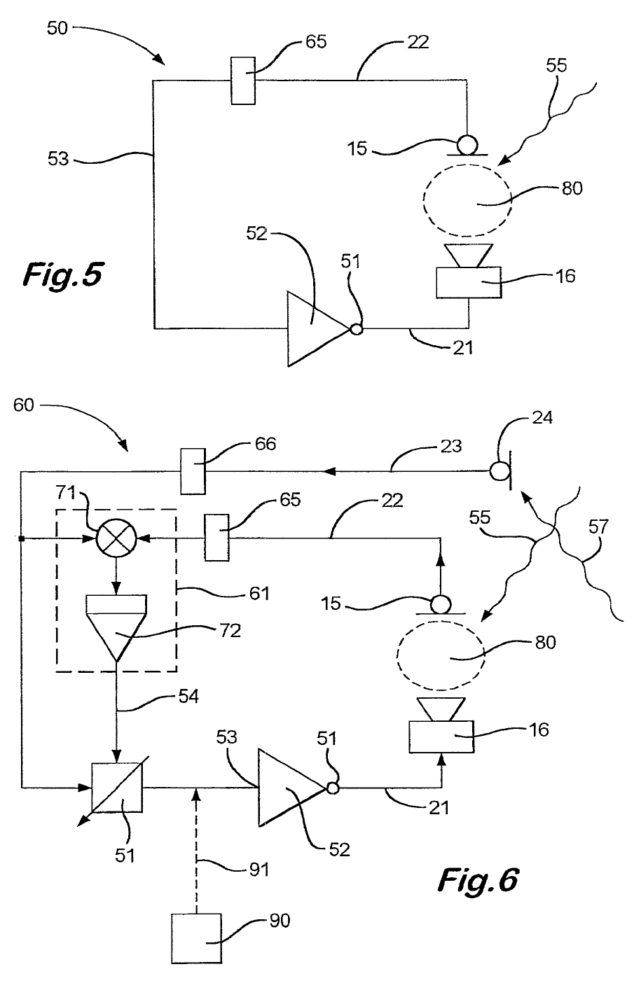

FIG. 5 is a circuit diagram of a basic cancellation system for use in the preferred embodiment, and

FIG. 6 is a circuit diagram of a more complex cancellation system for use in the preferred embodiment.

In FIG. 1, a person 10 who may be a driver or a passenger of a vehicle (not shown) is seated in a seat 12. A headrest 14 is moveably attached to the top of the seat and is disposed behind the persons head. The location of the headrest is such that a sound measuring device 15 can remotely measure the sound levels proximate the person's ear 18. A sound cancellation device 16 is also disposed in the headrest. The sound cancellation device is configured to generate a cancellation signal of approximately an equal and opposite polarity to the signal measured by the sound measuring device and to transmit this cancellation signal towards the ear 18. This has the effect of substantially reducing the amount of noise audible by the ear. The sound cancellation device is preferably a loudspeaker.

Also disposed in the headset is a tracking device 17 which is arranged to track the location of the ear 18 and to communicate this information to the sound measuring device 15. The tracking device may also communicate with the sound cancellation device 16. Various types of tracking devices, for example, a video tracking device could be used. The use of video devices to search, acquire and track a target is well known and as such not explained here in detail. The tracking device may require an initial set up step prior to normal operation.

In FIG. 2, where parts also appearing in FIG. 1 bear identical references, a pair of sound measuring devices 15a and 15b, a pair of sound cancellation devices 16a and 16b, and a pair of tracking devices 17a and 17b, are shown. The sound measuring devices are arranged to measure the sound levels proximate each of the person's ears 18a, 18b. The tracking devices are arranged to track the movement of the head 11 and to communicate this movement to the sound measuring devices. The sound measuring devices are further arranged to be adjusted so that sound can be measured at substantially the same location, regardless of any movement of that location. Advantageously, this facilitates the continuous measurement and cancellation of noise.

In FIG. 3, where parts also appearing in FIGS. 1 and 2 bear identical references, the basic noise cancellation system 20 is shown comprising sound measuring device 15 arranged to remotely measure the sound pressure variations in the vicinity of the ear canal 19. The measuring device is in communication with a processor 26 via a conductor 22. The processor is arranged to interpret the sound measured by the measuring device and to calculate the appropriate noise cancellation signal accordingly. The cancellation signal is then transmitted along a conductor 21 to the sound cancellation device 16, where the appropriate cancellation sound is generated and transmitted towards the ear.

The cancellation system may also include a microphone 24 arranged to provide an independent measurement of the background noise. The microphone is in communication with the processor 26 via a conductor 23. The background noise information provided by the microphone is used by the processor to facilitate calculation of the noise cancellation signal. The processor 26 may include either of the circuits to be described in FIGS. 5 and 6.

In order for the sound measuring device 15 to remotely measure the sound in the vicinity of the ear canal, a local transduction process must occur whereby the pressure variations caused by sound is transformed into a signal which can be measured remotely by the measuring device. This may be achieved in a variety of ways. In the preferred embodiment the sound measuring device is used to remotely access the vicinity of the ear canal. This is preferably mounted in the headrest 14 located at the top of the seat 12. Alternatively, the sound measuring device can be mounted in part of the body of the vehicle, for example, in the vehicle's doors.

The sound measuring device 15 is preferably at optical device, such as an interferometer. The operation of interferometers is well known to the skilled person and as such not explained here in detail. As is well known, an interferometer has a light source. In the preferred embodiment of the present invention this light source is a laser.

As is well known, skin will vibrate when exposed to sound waves. In the present invention the interferometer is arranged to measure the vibrations in the skin proximate to the ear canal, thereby determining the sound present at that location. As will be clear to the skilled man, the path from the interferometer to the location on the skin where the measurement is made must be substantially free from obstructions, such as hair or clothing.

In FIG. 4, an ear 18 is shown comprising an ear canal 19, a pinna 42, a concha 44, and a cavum 46. In the preferred embodiment the transduction process is a natural transduction whereby sound causes vibration of the skin in the pinna, the concha, and/or the cavum. The tracking device 17 is arranged to search, acquire and track the position of the pinna, concha, and/or cavum. The tracking device is further arranged to communicate this tracking information to the sound measuring device 15 so that the sound measuring device can measure the sound at any or all of these locations on the ear. Advantageously, this natural transduction process is entirely unobtrusive to the driver or the passenger of the vehicle.

In an enhanced embodiment of the present invention, pressure sensitive paint is applied to the ear either directly or via a thin sheet disposed contiguous to the pinna, concha, and/or cavulm. This enhanced embodiment functions much the same way as the natural transduction process described above, however, the sound measuring device now detects vibrations of the skin covered with the pressure sensitive paint. Advantageously, this enhanced embodiment is more sensitive than if no pressure sensitive paint were used, and is also unobtrusive.

In an alternative to the preferred embodiment a small disk-shaped sensor is located proximate to the ear canal. The sensor is responsive to sound and vibrates accordingly. The sensor may be worn by the driver or passenger as a fashion accessory, for example, as earrings. Advantageously, this alternative embodiment provides greater sensitivity than the natural transduction process previously described.

In yet a further alternative embodiment of the present invention the small disc-shaped sensor is arranged to transduce sound pressure variations into a voltage and the sound measuring device is configured to remotely measure voltage in the vicinity of the ear canal.

Alternatively, the small disc-shaped sensor is arranged to transduce sound pressure variations into a magnetic field and the sound measuring device is configured to remotely measure via induction the magnetic fields in the vicinity of the ear canal.

As will be appreciated, the use of pressure sensitive paint or sensors helps to better define the sound measurement area. Advantageously, a better defined sound measurement area reduces the requirements of the tracking device.

In FIG. 5, where parts also appearing in FIGS. 1-3 bear identical references, the circuit diagram 50 provides the basic noise cancellation function using a simple feedback loop arrangement. The sound measuring device 15, which may be a microphone, is arranged to measure noise 55. The microphone is connected to a filter 65 via conductor 22. The filter is connected to an amplifier 52 via conductor 24. The amplifier is connected to an inverter 51 and then to a loudspeaker 16 via conductor 21. The filter functions to select which noise is passed on to the amplifier and the inverter and subsequently cancelled by the cancellation signal. The selection can be based on the spatial or spectral characteristics of the sound. For example, the filter may be a low pass filter allowing low frequency sound such as road noise to pass and subsequently be cancelled by the cancellation signal. High frequency sound such as voices in not passed by the filter and thus not cancelled by the cancellation signal. The inverter 51 functions to change the phase of the signal by 180 degrees which forms the cancellation signal. This cancellation signal is then transmitted by the loudspeaker as a sound wave towards area 80. The result is that in area 80 the noise measured by the microphone will be substantially reduced. As will be appreciated, the area 80 preferably corresponds to the location of one of the driver's or passenger's ears.

In FIG. 6, where parts also appearing in FIG. 5 bear identical references, the circuit diagram 60 provides a more advanced noise cancellation function. The circuit 60 also uses the known Howells-Applebaum cancellation loop. This circuit includes, in addition the features shown in FIG. 5, a correlator 61 comprising a multiplier 71 and an integrator 72. The microphone 15 is connected to the correlator via conductor 22. The correlator is connected to an automatic gain control 51 via conductor 54. The gain control is connector via conductor 53 to an amplifier 52. The amplifier is connected to the inverter 51 which is connected via conductor 21 to a loudspeaker 16. The gain control functions to adjusts the gain and phase as a function of frequency of the signal output from the correlator. This signal is then inverted 180 degrees by the inverter to form the cancellation signal which is then transmitted by the loudspeaker as a sound wave towards area 80. The result is that in area 80 the noise measured by the microphone 15 will be substantially reduced.

The circuit may include a further microphone 24 which functions to independently measure the background sound 57. The further microphone is coupled to the correlator 61 via conductor 23. Disposed between the further microphone 24 and the correlator is a second filter 66. This filter functions to select which background noise is passed on to the correlator and subsequently cancelled by the cancellation signal. The filter 66 functions the same way as the filter 65 described above.

As will be appreciated, the gain control 51 may also include a delay means which advantageously compensates for any time delays caused by the use of two spatially distinct microphones.

The filters 65 and 66 may differ depending on the application. For example, the filters for a passenger's cancellation apparatus may be arranged such that all noise in cancelled. However, the filters for a driver's cancellation apparatus may be arranged such that all noise except for sirens, horns and the like are cancelled.

In an enhancement to the circuit 60, an independent signal, such as that from a radio 90, is input via conductor 91. This signal in summed with the output of the correlator and transmitted via the loudspeaker to the area 80. Advantageously, this allows for wanted signals, such as music from the radio to be efficiently transmitted to the area 80.

As will be appreciated by those skilled in the art, various modifications may be made to the embodiments hereinbefore described without departing from the scope of the present invention. For example, the apparatus and method could be used to reduce noise in a house or office, as well as in vehicles. The vehicle may be a road vehicle or an aircraft. The present invention may be used for safety purposes as well as for comfort. For example, certain types of vehicles such as tanks and tractors could use the present invention to reduce harmful levels of noise.

As will be appreciated by the skilled reader, references to light sources and light generally will, in the context of the present application, include ultraviolet and infra red light, as well as visible light, and similar radiation.

The present invention may be applied to observers other than human observers, such as animals or inanimate observers. In the case of animals, due consideration must be given to the anatomy of the observer, particularly if video tracking of the ear is used. Such applications may be useful, for example, in calming farm animals when restrained in stressful situations, such as milking or shearing, or in transit. Inanimate observers could include, for example, a sound transducer mounted in a baby seat for a car. It would be undesirable to mount a transducer to the baby directly, but by mounting a transducer directly in the car seat, the advantages of the invention may be achieved. Other examples include recording or telecommunications apparatus such as a tape recorder or mobile telephone. It may be desirable to reduce ambient noise at the location of such equipment, but undesirable to hard wire such equipment to a noise cancelling system such as may be found in a vehicle.

As described in the preceding description, the transducer may transmit a signal to a measuring device in any of a number of ways. Among these ways are light, voltage (electric field), magnetic field, which are linked in that no physical connections such as optic fibre, metallic wire or the like is required between the transducer and the measuring circuit. In the appended claims, the term “wire-less” is used to encompass all of these, and other, means of transmission between transducer and measuring circuit wherein no physical connection is required.

1. Apparatus for reducing noise in an area, comprising a transducer disposed at a first location and arranged to transduce sound substantially in said area into a signal which is measurably by a measuring device, said measuring device being disposed at said first location or at a second location and coupled to a sound cancellation device, said sound cancellation device being configured to generate a cancellation signal of approximately an equal intensity and opposite polarity to said measurably signal and to transmit said cancellation signal to said area thereby substantially reducing the amount of noise audible in said area.

2. Apparatus as claimed in claim 1, wherein said first location is in or proximate to said area.

3. Apparatus as claimed in any preceding claim, wherein said second location area is remote from said area.

4. Apparatus as claimed in any preceding claim, wherein said area is proximate to a human ear.

5. Apparatus as claimed in any preceding claim, wherein said transducer is human skin.

6. Apparatus as claimed in claim 5, wherein said human skin is a part of a human pinna.

7. Apparatus as claimed in any of claims 5-6, wherein said transducer includes pressure sensitive paint.

8. Apparatus as claimed in any of claims 1-4, wherein said transducer is a sensor.

9. Apparatus as claimed in claim 8, wherein said sensor is arranged to generate a voltage in response to sound.

10. Apparatus as claimed in claim 8, wherein said sensor is arranged to generate a magnetic field in response to sound.

11. Apparatus as claimed in any of claims 1-8, wherein said measuring device is an optical device.

12. Apparatus as claimed in claim 11, wherein said optical device is an interferometer.

13. Apparatus as claimed in claim 12, wherein said interferometer comprises a laser as a light source.

14. Apparatus as claimed in claim 9, wherein said measuring device is arranged to measure said voltage.

15. Apparatus as claimed in claim 10, wherein said measuring device is arranged to measure said magnetic field.

16. Apparatus as claimed in any preceding claims, wherein said apparatus further comprising a tracking device arranged to search for said transducer, to acquire a location of said transducer, and to track said location of said transducer, said tracking device being further arranged to communicate said location of said transducer to said measuring device.

17. Apparatus as claimed in claim 16, wherein said tracking device is disposed in a headrest.

18. Apparatus as claimed in any of claims 16-17, wherein said tracking device is a video tracking device.

19. Apparatus as claimed in any preceding claim, wherein said apparatus further comprising a further measuring device disposed remote from said area and arranged to measure background noise proximate to said area, said background noise being communicated to said sound cancellation device to facilitate reducing the amount of noise audible in said area.

20. Apparatus as claimed in claim 19, wherein said further measuring device is a microphone.

21. Apparatus as claimed in any preceding claim, wherein said apparatus further comprising a filter disposed between said measuring device and said cancellation device and arranged to pass a range of frequencies, thereby enabling said apparatus to cancel noise based on a frequency of said noise.

22. Apparatus as claimed in any preceding claim, wherein said area is in a vehicle.

23. A method for reducing noise in an area, the method comprising the steps of:
transducing sound in said area into a signal,
measuring said signal from a location remote from said area,
generating a signal of approximately an equal intensity and opposite polarity to said measured signal, and
transmitting said generated signal to said area, thereby substantially reducing the amount of noise audible in said area.

24. A method as claimed in claim 23, comprising the further step of measuring background sound remote to said area, and using said measurement of background sound to facilitate the reducing the amount of noise audible in said area.

25. Apparatus for reducing noise as hereinbefore described with reference to the accompanying figures.

26. Apparatus for reducing noise in an area proximate an ear of an observer, comprising:
a transducer arranged to transduce noise sound substantially in said area into a signal;
a measuring device arranged to measure the signal from the transducer;
a sound cancellation device configured:
to receive information from the measuring device;
to generate a cancellation sound of approximately an equal intensity and opposite polarity to said noise sound; and
to transmit said cancellation sound to said area thereby substantially reducing the amount of noise sound audible in said area by said observer,
characterised in that:
the transducer is mounted on the body of the observer,
the measuring device is remote from the transducer, and
the transducer wire-lessly transmits the signal, representing sound in the vicinity of the ear canal, to the measuring device.

27. Apparatus according to claim 26 wherein the wire-less transmission of the signal takes the form of light reflected from the transducer, and said measuring device is an optical device.

28. Apparatus as claimed in any of claims 26-27, wherein said transducer includes pressure sensitive paint.

29. Apparatus according to claim 28 wherein the transducer comprises a pressure sensitive paint applied to the skin of the observer.

30. Apparatus as claimed in any of claims 26-27, wherein said transducer is human skin.

31. Apparatus according to claim 29 or claim 30 wherein the skin forms part of the ear of the observer.

32. Apparatus as claimed in claim 31, wherein said skin is a part of a human pinna; concha; and/or cavum.

33. Apparatus as claimed in any of claims 27-32, wherein said optical device is an interferometer.

34. Apparatus as claimed in claim 33, wherein said interferometer comprises a laser as a light source.

35. Apparatus as claimed in claim 26, wherein said transducer is a sensor.

36. Apparatus as claimed in claim 35, wherein said sensor is arranged to generate a voltage in response to sound, and said measuring device is arranged to measure said voltage.

37. Apparatus as claimed in claim 35, wherein said sensor is arranged to generate a magnetic field in response to sound, said wireless transmission is a magnetic field, and said measuring device is arranged to measure said magnetic field.

38. Apparatus according to any of claims 35-36 wherein the transducer comprises a sensor embedded in an item of jewellery for wearing on the ear of the observer.

39. An item of jewellery, for wearing on or proximate the ear, comprising a transducer for use in a system according to any of claims 26-38.

40. Apparatus, or an item of jewellery according to claim 38 or claim 39, wherein the item of jewellery is in the form of an earring.

41. Apparatus as claimed in any of claims 26-38, or 40, wherein said apparatus further comprises a filter disposed between said measuring device and said cancellation device and arranged to pass a range of frequencies, thereby enabling said apparatus to cancel noise sound based on a frequency of said noise.

42. Apparatus as claimed in any of claims 26-38, 40 or 41, wherein said apparatus further comprising a tracking device arranged to search for said transducer, to acquire a location of said transducer, and to track said location of said transducer, said tracking device being further arranged to communicate said location of said transducer to said measuring device.

43. Apparatus as claimed in claim 42, wherein said tracking device is disposed in a headrest.

44. Apparatus as claimed in any of claims 42-43, wherein said tracking device is a video tracking device.

45. Apparatus as claimed in any of claims 25-38, 40-44, wherein said apparatus further comprises a further measuring device disposed remote from said area and arranged to measure background noise proximate to said area, said background noise being communicated to said sound cancellation device to facilitate reducing the amount of noise audible in said area.

46. Apparatus as claimed in claim 45, wherein said further measuring device is a microphone.

47. A method for reducing noise in an area proximate an ear of an observer, the method comprising the steps of:
transducing noise sound in said area into a signal,
measuring said signal
generating a sound of approximately an equal intensity and opposite polarity to said measured sound, and
transmitting said generated sound to said area, thereby substantially reducing the amount of noise audible in said area, characterised in that the method further comprises:
using a transducer mounted on the body of the observer; and wire-lessly transmitting the signal from the transducer to a remote measuring device.

48. A method according to claim 47 wherein the wire-less transmission of the signal is achieved by the reflection of light from the transducer, for measurement in an optical device.

49. A method as claimed in any of claims 47-48, wherein said reflection comprises reflection from pressure sensitive paint.

50. A method according to claim 49 further comprising the step of applying the pressure sensitive paint to the skin of the observer, for use as the transducer.

51. A method as claimed in any of claims 47-48, wherein human skin is used as the transducer.

52. A method according to claim 50 or claim 51 wherein the skin of the ear of the observer is used as the transducer.

53. A method as claimed in claim 52, wherein the skin of a human pinna; concha; and/or cavum, is used as the transducer.

54. A method as claimed in any of claims 47-53, wherein the light is measured in an interferometer.

55. A method as claimed in claim 54, wherein a laser is used as a light source for emitting light for reflection by the transducer.

56. A method as claimed in claim 47, wherein said transducer is a sensor.

57. A method as claimed in claim 56, wherein said sensor generates a voltage in response to the sound, and a measuring device measures said voltage.

58. A method as claimed in claim 56, wherein said sensor generates a magnetic field in response to the sound, the wireless transmission is performed by a magnetic field, and a measuring device measures said magnetic field.

59. A method according to any of claims 56-58 wherein the transducer is embedded in an item of jewellery for wearing on the ear of the observer.

60. A method as claimed in any of claims 47-59, further comprising filtering between a measuring device and said cancellation device to pass a range of frequencies, thereby enabling said apparatus to cancel noise sound based on a frequency of said noise.

61. Apparatus as claimed in any of claims 47-60, further comprising tracking by searching for said transducer, acquiring a location of said transducer, tracking said location of said transducer, and communicating said location of said transducer to said measuring device.

62. Apparatus as claimed in claim 61, wherein said tracking is performed by a video tracking device.

63. A method as claimed in claims 47-62, comprising the further step of measuring background sound remote to said area, and using said measurement of background sound to facilitate the reducing the amount of noise audible in said area.

64. Apparatus for reducing noise as hereinbefore described with reference to the accompanying figures.
